(19) United States
(12) Patent Application Publication
(10) Pub. No.: US 2026/0202399 A1
(43) Pub. Date: Jul. 16, 2026

(54) ATTACHING A CELL-SPECIFIC IDENTIFIER TO NUCLEIC ACIDS AND EXPRESSED PROTEINS OF A CELL

(71) Applicant: Trustees of Dartmouth College, Hanover, NH (US)

(72) Inventors: Jiwon Lee, Hanover, NH (US); Nicholas Curtis, Wilder, VT (US); Tae-Geun Yu, Hanover, NH (US)

(21) Appl. No.: 19/444,035

(22) Filed: Jan. 8, 2026

Related U.S. Application Data
(60) Provisional application No. 63/744,007, filed on Jan. 10, 2025.

(51) Int. Cl. G01N 33/543; C12N 15/10; G01N 33/53

(52) U.S. Cl. CPC G01N 33/54313; C12N 15/1055; G01N 33/5308

(57) ABSTRACT

Methods, kits and compositions for attaching a cell-specific identifier to nucleic acids and expressed proteins of a cell. A solid support has protein capture probes and polynucleotide capture probes attached to a surface of the solid support which comprise a cell-specific identifier. The protein capture probe comprises a protein capture tag, and the polynucleotide capture probe comprises a polynucleotide capture tag.

### CROSS-REFERENCE TO RELATED APPLICATIONS

This application claims the benefit of U.S. Provisional Patent Application No. 63/744,007, filed on Jan. 10, 2025, which is incorporated by reference herein in their entirety.

### SEQUENCE LISTING

The computer-readable Sequence Listing submitted on Mar. 24, 2026, and identified as follows: 16,030 bytes ST.26 XML document file named “Sequence_Listing.xml,” created Feb. 24, 2026, is incorporated herein by reference in its entirety.

### FIELD OF THE INVENTION

The present invention relates to methods of attaching identifiers or barcodes to nucleic acids and proteins from biological samples.

### BACKGROUND

Protein engineering commonly includes the use of display-based screening systems, exemplified by yeast and phage displays, to evaluate different variants of a protein of interest. To identify desirable variants from a large library, a physical connection between that protein and its corresponding gene (phenotype-genotype linkage) is generally relied upon. This connection allows for the identification of the DNA sequence encoding the variant after screening and selection. Display-based screening systems have become standard practice in antibody engineering, proving robust and highly effective, leading to the rapid and straightforward discovery of antibodies that bind to specific targets.

However, there are still drawbacks and difficulties in display-based screening. For instance, in order to establish a phenotype-genotype linkage within a library, mediators such as phages, ribosomes, or yeast cells are required. For example, yeast cells expressing a single gene will display a single type of antibody fragment on their surface, hence the term ‘display system’. The requirement of mediators which are much larger than antibody fragments—phages being about 1,000 times larger, and yeast cells about 10,000 times larger—introduces several challenges or limitations. The biochemical properties of antibodies displayed on phages or yeast cells are significantly affected by the mediators, preventing proper evaluation of the antibodies' native functionality. Moreover, display-based screening systems are generally unable to screen proteins of interest with multi-chain structures, requiring antibodies to be converted into single-chain antibody fragment formats, which alters their functionality. As a result, this approach only allows for the screening of the binding function of antibodies (Fab) and cannot assess Fc domain function, pharmacodynamics, stability, and other important characteristics. As a result, screening antibodies against difficult antigens (such as membrane proteins), evaluating their function in full-length antibody formats, or utilizing in vivo systems has been challenging with conventional display systems. These challenges hinder the development of promising antibody molecules for drug candidates, drug delivery platforms, and enhancing antibody functionality.

There remains a need for protein screening techniques which bypass the need for mediators and which are capable of using full-length antibodies in various assays.

### SUMMARY

The present disclosure provides methods for attaching a cell-specific identifier to a protein of interest expressed by a cell and to a nucleic acid from the cell encoding the protein. The methods comprise providing a cell that expresses a protein, and providing a solid support having protein capture probes and polynucleotide capture probes attached to a surface of the solid support. The protein capture probes and the polynucleotide capture probes on the solid support comprise a cell-specific identifier; that is, both types of capture probes have the same unique identifier or barcode. The protein capture probe comprises a protein capture tag, and the polynucleotide capture probe comprises a polynucleotide capture tag. The method also comprises forming a single-cell emulsion comprising the solid support and the cell; incubating the single-cell emulsion so that the protein capture tag binds to or forms a conjugate with the expressed protein and the polynucleotide capture tag binds to or forms a conjugate with the nucleic acid; and detaching the protein capture probe from the solid support.

### BRIEF DESCRIPTION OF THE DRAWINGS

FIGS. 1A, 1B and 1C illustrate an overall workflow for an embodiment of the present antibody screening platform using a cell-specific identifier.

FIG. 2A illustrates an embodiment of a method for generating a cell-specific identifier by a split and pool technique with for dual targeting. FIG. 2B shows analyses of the generated cell-specific identifiers.

FIG. 3A illustrates the attachment of a cell-specific identifier to an antibody with an intermediary tag. FIG. 3B shows analysis of the barcoded antibodies.

FIGS. 4A and 4B illustrate single-cell emulsification and attaching a cell-specific identifier to an antibody in an embodiment of the present methods.

The present teachings are best understood from the following detailed description when read with the accompanying drawing figures. The features are not necessarily drawn to scale.

### DETAILED DESCRIPTION

The present disclosure provides solutions to problems in protein screening arising from needing mediators and using full-length antibody formats. The present methods are beneficial for screening antibodies with desired functionalities or properties, allowing for better evaluation of those antibodies, even in in vivo systems. By labeling antibodies or other proteins with a unique identifier shared with nucleic acids encoding such proteins, the need for a physical genotype-phenotype linkage is eliminated, addressing a long-standing drawback. The present methods allow for easy identification of an antibody's sequence by using the cell-specific identifier as an index. It is expected that the present approach can overcome at least some of the persistent limitations of conventional protein screening techniques and antibody engineering platforms.

Since the physical linkage between genotype and phenotype required by display-based screening system is not required by the present methods, a greater variety of protein functions (enzymatic, regulatory, inhibitory, binding, and/or structural) can be analyzed and more types of assays can be performed. This is advantageous over existing display selection strategies (e.g., yeast and phage display) where selection is based upon binding interactions alone. Since binding does not necessary correlate with other functions, the use of display-based screening may lead to the selection of poor inhibitors that bind tightly to an antigen or receptor outside of its active site.

Before the various embodiments are described, it is to be understood that the teachings of this disclosure are not limited to the particular embodiments described, and as such can, of course, vary. The section headings used herein are for organizational purposes only and are not to be construed as limiting the subject matter described in any way.

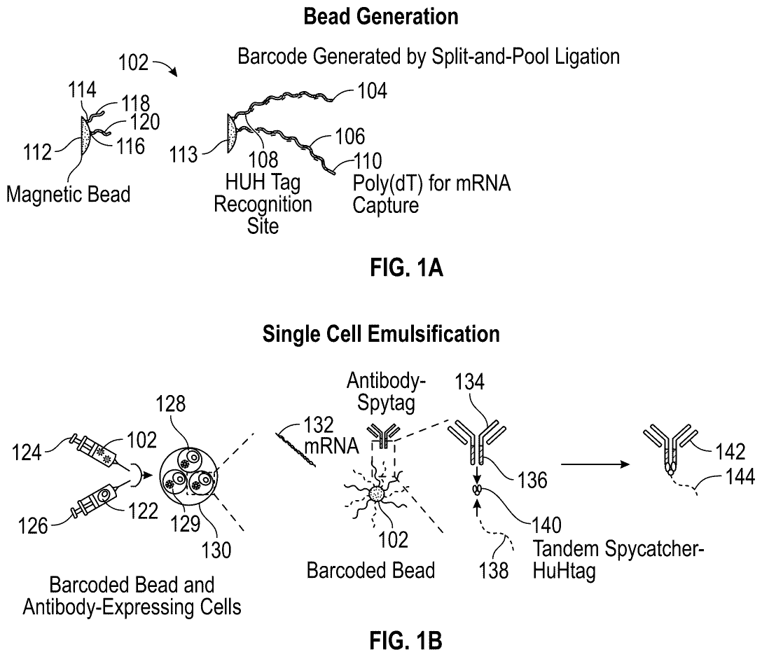

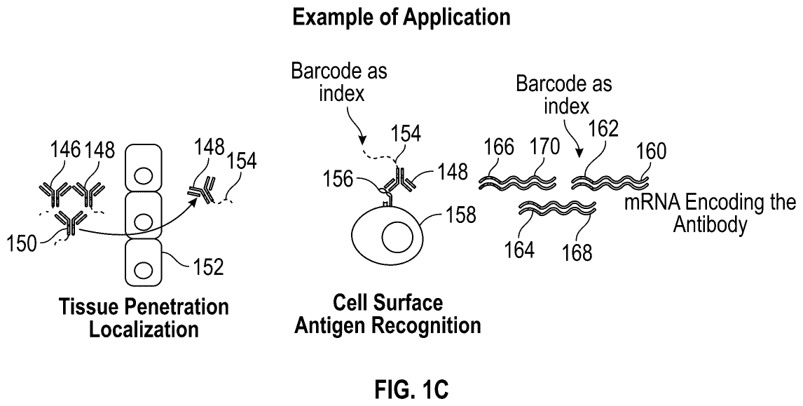

As one aspect, the present disclosure provides methods for attaching a cell-specific identifier to a protein expressed by a cell and to a nucleic acid from the cell. FIGS. 1A to 1C illustrate an overall workflow for a protein screening platform using a cell-specific identifier. The present methods are described with respect to development of antibody engineering platform technology by enabling barcoding of an antibody and its corresponding transcript (mRNA encoding the antibody) with a cell-specific identifier (e.g., the same short DNA barcode is attached to a protein expressed by the cell and to amplicons of nucleic acids from the same cell). It should be recognized that proteins other than antibodies can also be screened, and the following description with respect to barcoding an antibody is applicable to any protein of interest that can be expressed by a cell.

The present methods comprise providing a solid support 102 having protein capture probes 104 and polynucleotide capture probes 106 removably attached to a surface 113 of the solid support. The protein capture probes and polynucleotide capture probes on the solid support comprise a cell-specific identifier. The protein capture probe 104 comprises a protein capture tag 108, and the polynucleotide capture probe 106 comprises a polynucleotide capture tag 110. The protein capture tag 108 is adapted for binding to or forming a construct with the expressed protein directly or indirectly, and the polynucleotide capture tag is adapted for binding to or forming a construct with a nucleic acid encoding the expressed protein directly or indirectly. In some embodiments, the polynucleotide capture probes may comprise a homopolymeric sequence of deoxythymidines (poly(dT)) as a targeting group. In other embodiments, the polynucleotide capture probes may comprise a primer specific to a target nucleic acid.

FIG. 1A shows an embodiment of how the solid support 102 having protein and polynucleotide capture probes 104, 106 can be generated. A bead 112 has linking moieties 114, 116 on its surface which can bind to initial portions 118, 120 of the capture probes. The initial portions 118, 120 can be the same or different; in some embodiments, the initial portion 118 for the protein capture probe 104 differs from the initial portion 120 of the polynucleotide capture probe 106, to facilitate subsequent attachment of different segments. FIG. 1B shows an embodiment in which the solid supports 102 are combined with cells 122 in a manner which forms single-cell emulsion microdroplets 128 comprising one cell 122 per microdroplet. The solid supports 102 are fed from a solid support source 124 and the cells 122 are fed from a cell source 126. The emulsion microdroplets 128 comprise an aqueous phase 129 in an oil phase 130. As explained in more detail below, the cells 122 can be lysed within the emulsion microdroplets 128 so as to release mRNA transcripts 132 from cell 122. A protein 134 expressed by the cell 122 (e.g., an antibody) is also within the microdroplet 128. Capture tags on the protein capture probes 104 bind or form a conjugate with the protein 134 directly or indirectly, and targeting groups on the polynucleotide capture probes 106 bind or form a conjugate with the mRNA molecules 132 directly or indirectly.

In some embodiments, a capture tag binds directly, such as when a polynucleotide capture tag hybridizes with a complementary sequence in a mRNA molecule 132. In some embodiments, a capture binds indirectly, such as when a polynucleotide tag on a capture probe binds with an expressed protein tag on an expressed protein. In FIG. 1B, the protein 134 comprises an expressed protein tag 136 which binds with a protein capture tag 138 via an intermediary tag 140, thereby forming a barcoded protein 142 having a cell-specific identifier 144.

FIG. 1C shows an embodiment in which a plurality of barcoded antibodies 146, 148, 150 are used in an assay which evaluates their penetration and/or localization across a membrane 152. The barcoded antibodies 146, 148, 150 are contacted with a first side of a membrane 152 (such as a natural or synthetic tissue membrane), and passage and/or amount of the barcoded antibodies on the opposite side of the membrane is determined. In FIG. 1C, barcoded antibody 148 penetrates the membrane 152 whereas barcoded antibodies 146, 150 do not. Barcoded antibody 148 can be further evaluated for its binding to an antigen of interest 156 on an antigen-presenting cell 158 (or other type of cell), and/or for other antibody properties, and its cell-specific identifier 154 can be sequenced. By finding the same cell-specific identifier 162 on amplicons 160 of mRNA transcripts from the same cell 158, the sequences of the mRNA encoding the barcoded antibody 148 can be readily determined. In some embodiments, the cell-specific identifiers 164, 166 are used to identify amplicons 168, 170 of mRNAs for barcoded antibodies 146, 150 which did not penetrate the membrane 152, or did not bind antigen 156 to a desired or sufficient degree, or which were not satisfactory for some other reason.

### Solid Support

The present methods, compositions, and kits can employ any solid suitable for providing a surface for the capture probes. The capture probes are attached directly or indirectly to the surface of a solid support, such as a bead, a flowcell, a slide, a plate, a device comprising one or more microfluidic channels, or a column. Solid supports can be made from polymer resins (e.g., (methyl)acrylates, polystyrenes, polyacrylamides, polyethylene glycols) and/or paramagnetic/magnetic materials. “Bead”, as used herein, includes particles, microbeads, nanoparticles, and any other substantially spherical solid supports. In some embodiments, the solid support is a magnetic bead. In some embodiments, the bead has a diameter of from about 0.5 μm to about 100 μm, from about 1 μm to about 50 μm.

In some embodiments, the solid support (e.g., bead) has one or more linking moieties on its surface adapted for attachment of the capture probes. The linking moieties can comprise one or more reactive groups that form a bond with reactive group(s) on the capture probes or initial portions of capture probes. For example, the solid support can have a coating that adheres to a core, wherein the coating comprises one or more linking moieties. The linking moiety can be any functional group that reacts with a reactive group on a capture probe. The linkage can be formed through covalent bonds using chemically reactive groups or through non-covalent bonds, such as protein-ligand interactions like the biotin-streptavidin complex. Examples of linking moieties include photo-cleavable linkers, enzymatically cleavable linkers, and disulfide bond linkers. In some embodiments, the linking moiety comprises a spacer between the surface of the solid support and the functional group. Examples of such spacers include polyethylene glycol (PEG) and other polyalkylene oxides. In some embodiments, the linking moiety comprises a nucleic acid or a peptide comprising a sequence recognized by an enzyme. For example, the linking moiety can comprise a peptide cleavable by an endogenous or exogenous enzyme.

### Synthesis of Capture Probes on Solid Supports

In some embodiments, the present disclosure provides a method for synthesizing a set of solid supports such as beads with unique cell-specific identifiers on each solid support. Each of the capture probes on an individual solid support has the same cell-specific identifier, and each of the solid supports within a set has a different cell-specific identifier than the other solid supports in the set. In some embodiments, each of the capture probes on a solid support has a cell-specific identifier which is substantially unique in the set, meaning that it is intended to be and is (for practical purposes) unique with respect to cell-specific identifiers on every other solid support in the set.

Capture probes comprising cell-specific identifiers can be synthesized in various ways involving the addition of nucleotides to a growing polynucleotide strand. In some embodiments, capture probes comprising a cell-specific identifier are synthesized using a split-and-pool process, which employs cycles of ligation of relatively short DNA segments to the ends of the probes. The cell-specific identifier can be prepared by split-and-pool combinatorial ligation or by split-and-pool enzymatic ligation reaction. By way of example, beads comprising linking moieties are dispensed into multiple partitions (for example, 96 or 384 wells) in which a segment is ligated to the ends of the probes on all the beads in the partition. Different segments are used in different partitions. After ligation, the beads are pooled, then split again in partitions. Eventually, each bead stochastically has different combinations of ligated DNA segments, and the combination of ligated segments serves as an identifier unique to each bead.

The cell-specific identifiers can be synthesized on the surface of the beads or other solid supports by using a split-and-pool technique where, in each cycle of polynucleotide synthesis, the beads of the set are split into partitions and subjected to reactions adding an oligonucleotide comprising one or more nucleotides of the identifier, followed by pooling and mixing the beads after completing such reactions. Different oligonucleotides are added to different partitions in order to provide the desired complexity of sequences. Then this split-pool process is repeated in one or more cycles of ligation, to produce a combinatorially large number of distinct cell-specific identifiers in the set. The polynucleotide synthesis can be conducted in a step-wise fashion, wherein one or more nucleotides of the identifier are added to the capture probes in each cycle. Examples of polynucleotide synthesis techniques include, but are not limited to, solid-phase chemical synthesis using phosphoramidite building blocks derived from protected 2′-deoxynucleosides (dA, dC, dG, and dT), ribonucleosides (A, C, G, and U), or chemically modified nucleosides such as locked nucleic acid (LNA). To obtain the capture probe, the chemical building blocks can be sequentially coupled to the growing capture probe in a predetermined or random order.

The solid supports comprise protein capture probes and polynucleotide capture probes to barcode a protein expressed by a cell and nucleic acids from the same cell with the same cell-specific identifier. Each solid support contains at least two types of capture probes, one for barcoding a protein expressed by the cell and another for capturing and barcoding mRNA from the same cell. The two or more types of capture probes differ with respect to their capture tags.

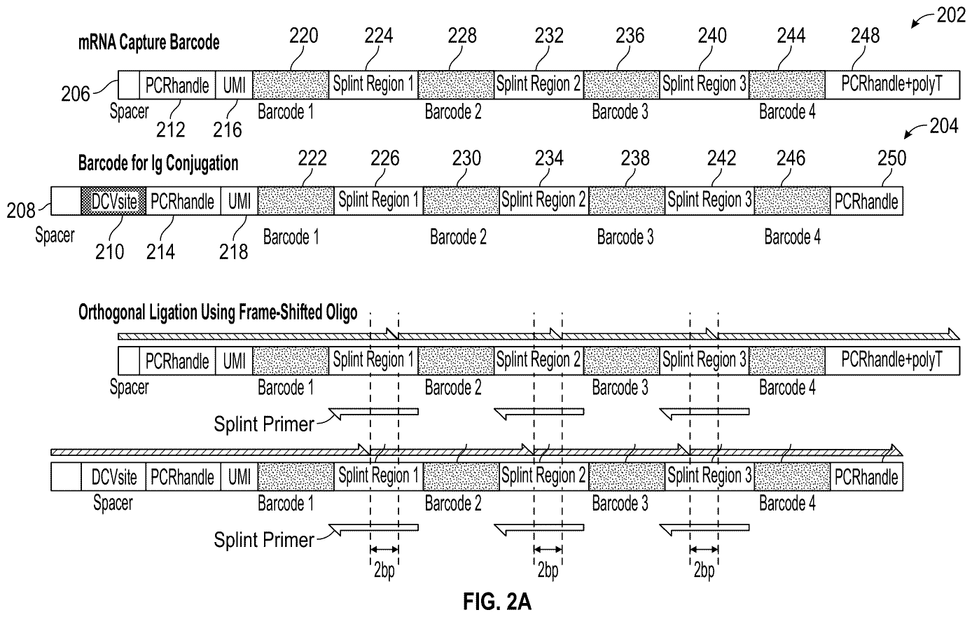

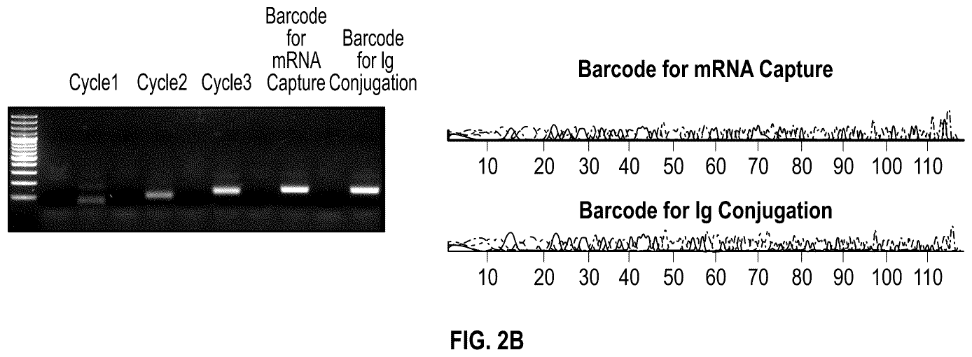

In some embodiments, the protein capture probes and polynucleotide capture probes are orthogonally synthesized so that they have different capture tags or other differences in their structures while still having the same cell-specific identifier sequence. FIG. 2A illustrates the utilization of frame-shifted short DNA segments to synthesize two types of capture probes having the same cell-specific identifier. As shown therein, the polynucleotide capture probes 202 and protein capture probes 204 are framed-shifted compared to each other, so they are ligated orthogonally. The capture probes 202, 204 shown in FIG. 2A in 5′ to 3′ orientation comprise spacers 206, 208 which can be a linking moiety attached to a solid support or can be adapted for binding to such a linking moiety. Protein capture probe 204 comprises a protein capture tag 210 adapted for binding or forming a construct directly or indirectly with an expressed protein. The capture probes 202, 204 next comprise a primer binding site 212, 214 (or “PCR handle”) which provides a known sequence for a primer to bind to, for amplification and/or sequencing. The capture probes 202, 204 comprise unique molecular identifiers (UMI) 216, 218, first cell-specific identifier segments 220, 222, and first splint regions 224, 226. The splint regions result from the orthogonal segment ends which are used for ligation of the identifier segments; the splint regions are interspersed in the cell-specific identifier but they are not part of the barcode sequence itself. Protein capture probes and polynucleotide capture probes can be synthesized orthogonally by utilizing different sequences in the splint region between the two, or by using frame-shifted DNA segments. Ultimately, protein capture probes and polynucleotide capture probes each acquire a unique barcode sequence for each individual solid support. For example, the segments can have the following structures:

In case of using different splint region sequence,
Protein capture probe segment XXXXTCACTAGA
Polynucleotide capture probe segment XXXXGATGACAG

In case of using frame-shifted DNA segments,
Protein capture probe segment XXXXTCACTAGA
Polynucleotide capture probe segment XXXXTCACTA

where XXXX represents a barcode/identifier region comprising various nucleotides Through such a process, a selected ratio of two different type of capture probes can be synthesized orthogonally, but with same cell-specific identifier sequence. The polynucleotide capture probe 202 and the protein capture probe 204 have the same identifier sequence, but different functionality on the strand. The capture probes 202, 204 comprise second cell-specific identifier segments 228, 230, second splint region 232, 234, third segments 236, 238, third splint regions 240, 242 and fourth segments 244, 246. The capture probes 202, 204 comprise another primer binding sequence 248, 250, for amplification and/or sequencing.

In some embodiments, the primer binding sequence 248 of the polynucleotide capture probe 202 can be a poly(dT) sequence, so that it can also serve as a polynucleotide capture tag. In other embodiments, the primer binding sequence 248 is a different sequence from the polynucleotide capture tag. When the polynucleotide capture barcode has poly(dT) at the 3′ end as its polynucleotide capture tag, it can capture mRNA transcripts by hybridizing to poly(A) at the 3′ end of the mRNA molecules. After such hybridization, reverse-transcription PCR or other amplification technique can be performed to generate the complement of the mRNA molecule 3′ to the poly (dT). Complements of RNA are easily used by NGS systems to determine RNA sequences.

In some embodiments, the protein capture tag 210 of the protein capture probe 204 is a HUH-tag recognition site (labeled as “DCV” site in FIG. 2A), which is recognized by HUH-tag and covalently conjugated to (or indirectly binds) antibody. In some embodiments, a SpyCatcher-HUH-tag construct is employed as an intermediary tag. This enables efficient one-step site-specific conjugation between a Spy-Tagged antibody and a DNA barcode within a single-cell emulsion. The terms “SpyTag” and “SpyCatcher” refer to a binding pair that forms a covalent bond, which are described in Zakeri et al., Proc Natl Acad Sci USA. 2012 Mar. 20; 109(12):E690-7. SpyTag is an expressible peptide that will form a spontaneous amide bond with its partner SpyCatcher under a wide range of conditions.

FIG. 2B shows results of testing the split-and-pool synthesis method, in which DNA barcode segments were ligated step-by-step (Cycles 1, 2 and 3 indicate the number of cycles for ligation of DNA barcode segments). These capture probes 202, 204 can be synthesized orthogonally and thus can be amplified separately. The polynucleotide capture probes and protein capture probes can be synthesized through split-and-pool ligation using splint primers and then amplified with PCR handles. To verify each sequence, different PCR handles were used for the polynucleotide capture barcode and protein capture barcode, and the resulting amplicons were subjected to Sanger sequencing. Sequencing confirmed that the resulting barcodes for capture of transcripts and the protein (e.g. expressed antibody) were identical.

The overall structure of the cell-specific identifier, such as how many ligation cycles are used or the base pair for the frame shift, can be selected by the user and may differ depending on the purpose of experiment. For example, the number of cycles of ligation to form the cell-specific identifier can be changed for increased or decreased diversity of the identifier.

In some embodiments, the spacers 206, 208 could be modified to be cleavable linkers. In some embodiments, the HUH-tag recognition site could be positioned at either the 5′ or 3′ end of the barcode, depending on the experiment's objective.

### Protein Capture Tags And Polynucleotide Capture Tags

As explained above, the solid supports have protein capture probes and polynucleotide capture probes attached to a surface. The protein capture probes and polynucleotide capture probes on an individual bead comprise the same cell-specific identifier but different targeting groups. The protein capture probe comprises a protein capture tag, and the polynucleotide capture probe comprises a polynucleotide capture tag. The protein capture tag binds directly or indirectly to the expressed protein, and the polynucleotide capture tag binds directly or indirectly to a nucleic acid encoding the expressed protein.

In some embodiments, when the nucleic acid sought for the experiment comprises mRNA, the polynucleotide capture tag comprises poly(dT). In some embodiments, the polynucleotide capture tag comprises a target-specific region that hybridizes to a target sequence, such as a target gene sequence or a target transcriptome sequence.

In some embodiments, the protein capture tag comprises a reactive chemical moiety (such as reactive amine) that binds a lysine residue of the protein of interest. In some embodiments, the protein capture tag binds the expressed protein via click chemistry, such as by binding of alkynes and azides. Examples of alkynes and azides binding via click chemistry include copper-catalyzed reaction of an azide and alkyne to form a triazole (Huisgen 1,3-dipolar cycloaddition) and strain-promoted azide alkyne cycloaddition (SPAAC).

In some embodiments, the protein capture tag comprises a HUH-tag recognition site, and the method further comprises binding an intermediary tag comprising a HUH-tag to the expressed protein of interest. In some embodiments, an intermediary tag comprises two binding members which are from two different binding pairs, wherein a first binding member is a binding pair with an expressed protein tag and the second binding member is a binding pair with a protein capture tag. For example, the intermediary tag can be a SpyCatcher-HUH-tag construct, where SpyCatcher is a member of a first binding pair (with SpyTag), and HUH-tag is a member of a second binding pair (with a HUH-tag recognition site). In some embodiments, an intermediary tag is a construct comprises two binding members in tandem. In some embodiments, such a tandem construct is used as an intermediary tag for simple and fast one-step conjugation between the expressed protein and the protein capture probe.

In some embodiments, the present methods employ an intermediary tag that includes the reciprocal binding partner of the expressed protein tag (that is, the other binding partner of a first binding pair). For example, the expressed protein tag can comprise a SpyTag, and the intermediary tag can comprise a SpyCatcher domain. In some embodiments, the intermediary tag also includes a binding partner of a second binding pair (that is, a binding pair that is different from the first binding pair). The binding partners of the second binding pair do not bind with the binding partners of the first binding pair. For example, the second binding pair can include a HUH-tag and a HUH-tag recognition site.

In some embodiments, the expressed protein tag is any binding partner suitable for covalent or non-covalent attachment to the expressed protein of interest.

In some embodiments, the expressed protein tag or the intermediary tag comprises a sequence-specific DNA binding protein or active domain thereof, such as a HUH-tag. A HUH-tag comprises an active domain of a HUH endonuclease, which is a nuclease comprising a HUH (histidine-hydrophobic amino acid-histidine) tag that can form covalent bonds with specific single-stranded DNA sequences. Additional information about HUH endonucleases can be found in Nagy et al. US 20220282259 A1. In some embodiments, the HUH-tag comprises a Duck Circovirus 2 (DCV) Rep protein or active domain thereof, and the polynucleotide capture tag comprises a DCV recognition site.

Examples of HUH-tags and HUH-tag recognition sites are shown in Table 1.

TABLE 1

| HUH-tags | HUH-tag recognition site |
|---|---|
| duck circovirus Rep protein | AAGTATTACCAGAAA (SEQ ID NO: 1) |
| fava bean necrosis yellow virus Rep protein | AAGTATTACCAGAAA (SEQ ID NO: 1) |
| porcine circovirus Rep protein | AAGTATTACCAGAAA (SEQ ID NO: 1) |
| RepB Streptococcus agalactiae | TGCTTCCGTACTACGACCCCCCA (SEQ ID NO: 2) |
| RepB Fructobacillus tropaeoli | TGCTTCCGTACTACGACCCCCCA (SEQ ID NO: 2) |
| conjugation protein TraI Escherichia coli | TTTGCGTGGGGTGTGGTGCTTT (SEQ ID NO: 3) |
| mobilization protein A Escherichia coli | CCAGTTTCTCGAAGAGAAACCG |
| | GTAAGTGCACCCTCCC (SEQ ID NO: 4) |
| nicking enzyme Staphylococcus aureus | ACGCGAACGGAACGTTCGCATA |
| | AGTGCGCCCTTACGGGATTTAAC (SEQ ID NO: 5) |

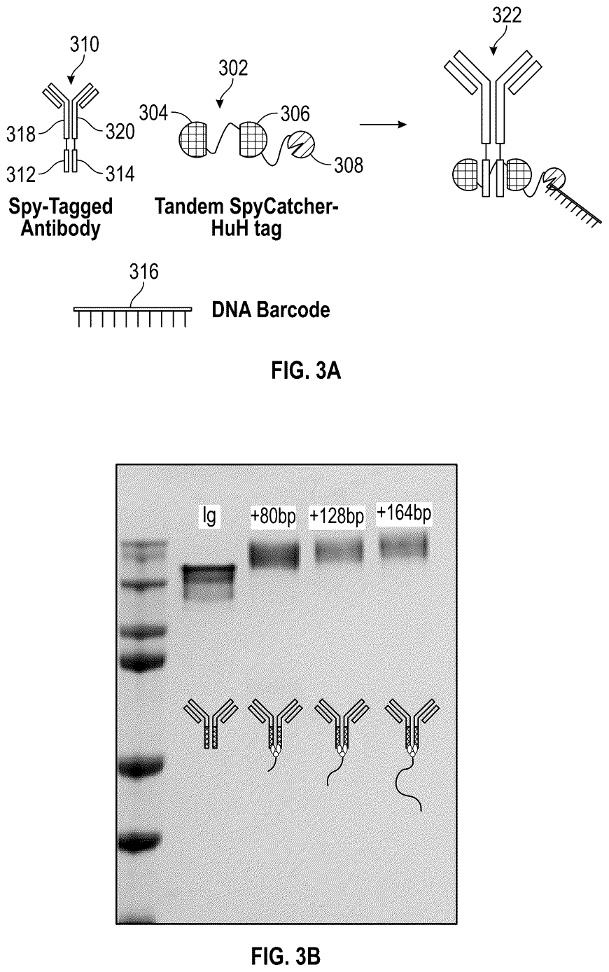

FIG. 3A illustrates the use of a tandem SpyCatcher-HUH-tag construct 302 as an intermediary tag. A tandem SpyCatcher-HUH-tag (TdSPH) construct 302 was created by genetically fusing two SpyCatcher domains 304, 306 with an HUH-tag 308. The SpyCatcher domains 304, 306 can be the same or different. The antibody 310 expressed by the recombinantly engineered cell comprises two SpyTags 312, 314 as expressed protein tags, which can also be the same or different from each other, so long as they are members of binding pairs with the SpyCatcher domains 304, 306. Each of the two SpyCatcher domains 304, 306 forms a covalent bond with one of the SpyTags 312, 314, and the HUH-tag 308 can form a covalent bond with a HUH-tag recognition site 316, which serves as the protein capture tag of the protein capture probe. The SpyTags 312, 314, being a small peptide, has negligible effects on the expression or biochemical properties of the protein 310.

In this embodiment, the SpyTags 312, 314 were genetically fused to the C-terminal of the antibody heavy chains 318, 320, resulting in two SpyTags 312, 314 tethered to the antibody's Fc domain. In other embodiments, SpyTags or other expressed protein tags can be attached to the protein in a separate reaction after expression. Using TdSPH, the SpyTagged antibody 310 binds with the protein capture tag 316 through simple incubation, forming a barcoded antibody 322. This technique proved to be highly efficient and robust. Additionally, the reaction is site-specific, without concerns about barcode number variation per antibody or steric hindrance. This embodiment enables a precise quantification after screening and minimizes functional interference with the antibody. Binding affinities of the antibody molecules to its target antigen, as well as to Fc binding proteins (protein G and Fc gamma receptor III) with and without the attached barcode were compared using biolayer interferometry. There was no significant alteration in binding affinity in the Fc or Fab regions.

FIG. 3B shows the efficient conjugation between the antibody and various lengths of DNA using SDS-PAGE gel analysis. The first lane is the protein ladder, while the second lane contains the native antibody without the barcode. The third, fourth, and fifth lanes represent antibodies conjugated with 80, 128, and 164 bp barcodes, respectively, via SpyTag and an intermediary tag. The size shifts observed between these lanes indicate the successful attachment of the barcode to the antibody.

### Immobilization Of Secreted Antibody On Cell Surface

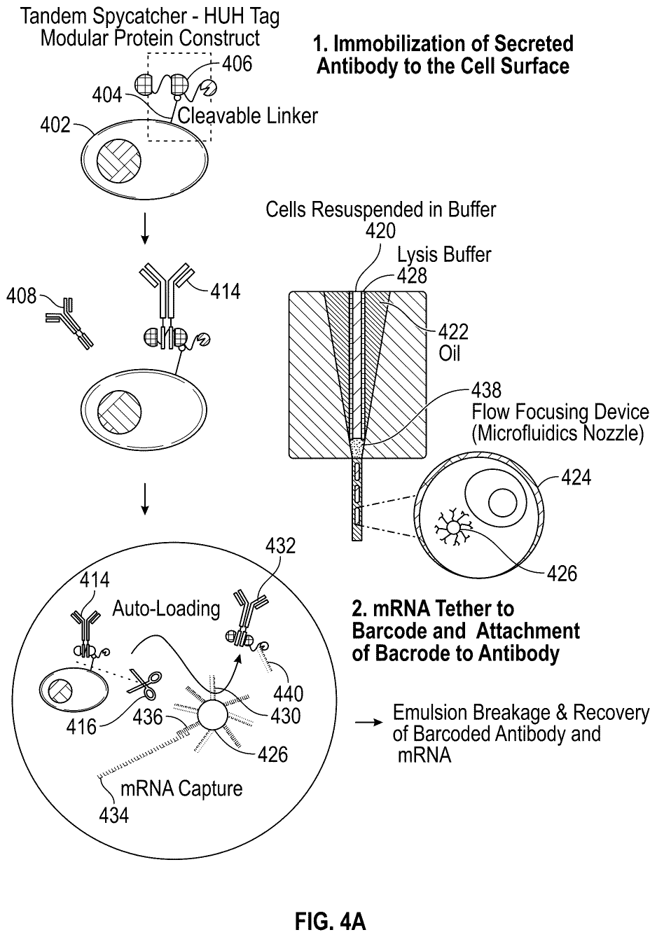

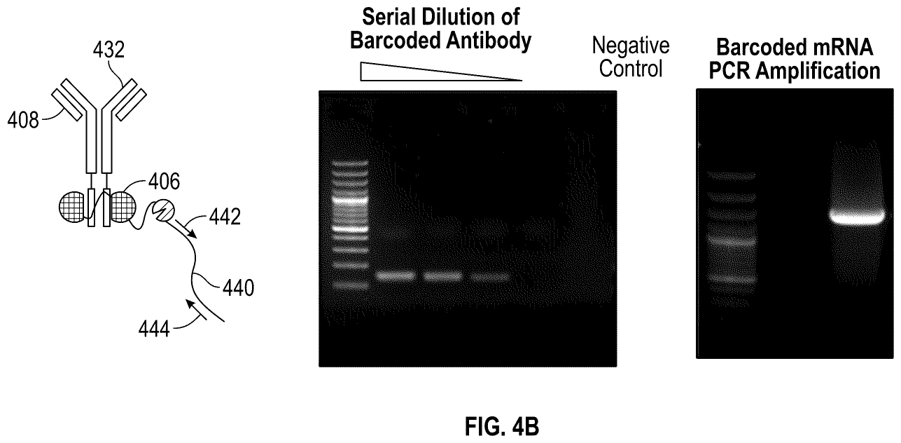

In some embodiments, to facilitate attachment of the protein capture probe to a secreted protein expressed by a cell within a single-cell emulsion microdroplet, the secreted protein can be immobilized on the cell surface before the cell is subjected to emulsification. FIG. 4A illustrates such an embodiment, in which TdSPH is used to immobilize secreted antibodies onto the cell surface. A cell 402 is genetically engineered to express a SpyTag-labeled protein of interest (e.g., an antibody) library. For intracellular proteins, the barcode attachment process can be achieved directly using the TdSPH module. For proteins secreted extracellularly, like antibodies, immobilization is facilitated by proximity-dependent capture through the TdSPH module, which is pre-immobilized on the cell surface through cleavable linker 404. In the depicted embodiment, the cell surface moiety 404 is a cleavable linker attached to an intermediary tag 406 which can bind to an expressed protein 408.

In some embodiments, TdSPH fused with an Avi-Tag, which is a short peptide having the sequence GLNDIFEAQKIEWHE (SEQ ID NO:6). When the Avi-Tag is combined with E. coli biotin ligase (BirA) and biotin N-hydroxy succinimide ester (NHS-biotin), the ligase catalyzes binding of biotin to the Avi-Tag, such that the TdSPH is biotinylated. The TdSPH-avi-tag is recombinantly expressed by the cell, linked by a cleavable linker comprising a site recognized by recombinant Tobacco Etch Virus (TEV) protease. The resulting TdSPH was immobilized on the cell surface by treating the cells with N-hydroxy succinimide biotin (NHS-biotin) and streptavidin. NHS-biotin is a cell-impermeable chemical that reacts with primary amines, such as lysine residues on cell surface proteins. When cells are treated with NHS-biotin, biotin moieties are covalently attached to the cell surface through an NHS-amine reaction. Subsequently, a biotinylated TdSPH-streptavidin complex is applied. Due to streptavidin's tetrameric structure, the complex binds efficiently to the biotinylated cell surface, facilitating downstream applications.

Alternatively, the antibody expressed by a cell can be immediately immobilized to the cell surface through the interaction between a SpyTag and TdSPH. This proximity ensures that each cell within the library secretes its own antibody variant, which is then immobilized on the corresponding cell. By incubating TdSPH-immobilized cells in the media for an incubation period, cells 412 comprising their expressed antibodies 408 (or other expressed protein of interest) on their surface.

### Forming A Single-Cell Emulsion Comprising One Solid Support And One Cell

For attachment of a cell-specific identifier from a solid support to an expressed protein and its corresponding mRNA transcript, cells expressing the protein of interest (typically a library of diverse cells expressing different variants of the protein) and a solid support as described herein can be co-encapsulated into an emulsion microdroplet droplet through a single-cell emulsification technique.

The cells are typically suspended in a buffer or other aqueous medium and subjected to single-cell emulsification. An aqueous phase 420 containing both cells 412 and beads is prepared and then emulsified into oil phase, typically by injecting it into a fast-moving stream of oil phase 422. The shear forces generated by the moving oil phase create microdroplets 424 as the aqueous phase 420 is injected into the stream, creating an emulsion with a low dispersity of droplet sizes. Each cell 412 is in its own microdroplet droplet along with one or more beads 426 conjugated with protein and polynucleotide capture probes. Uniformity of microdroplet size helps to ensure that individual droplets do not contain more than one cell. Exemplary microdroplet sizes are from about 10 μm to about 250 μm, alternatively from about 50 μm to about 150 μm, alternatively about 100 μm.

A water-in-oil single-cell emulsion that contains only one cell and one bead can be formed by any suitable technique. A water phase is prepared which contains cells and solid supports, suspended in a small volume of water. An oil phase can be formed from a suitable oil and a surfactant that can stabilize the emulsion. Examples include mineral oil and surfactants such as sorbitan monooleate (Span 80) or other non-ionic surfactants; lecithin or other natural surfactants; and sodium stearoyl lactylate or other ionic surfactants.

For emulsification, the aqueous phase is combined with the oil phase, such as by stirring vigorously or by using a microfluidic device or a homogenizer to ensure that aqueous microdroplets containing the cell and bead are uniformly dispersed in the oil. For forming and isolating single-cell emulsion microdroplets containing one cell and one bead, one may use techniques like flow cytometry or microfluidic sorting. Suitable devices which can be used to co-encapsulate single cells and beads into individual emulsion droplets include flow-focusing devices 438 which use a flow-focusing mechanism to create uniform droplets; T-junction microfluidic chips which generate droplets at the intersection of two microchannels, allowing control over droplet size; droplet generator chips which can produce picoliter-scale droplets at high rates, making them suitable for high-throughput applications; and microchannel emulsification devices which use microchannels to produce emulsions with high uniformity and control over droplet size. Other kinds of microfluidics can be used to emulsify the cells and beads together.

In some embodiments, the present method further comprises introducing a lysis reagent 428 into the single-cell emulsion, thereby inducing lysis of the cell and releasing nucleic acid from the cell. Cells can be lysed within the emulsion microdroplets 424 containing solid supports 426 comprising protein and polynucleotide capture probes, thereby releasing nucleic acid encoding the expressed protein from within the cell and facilitating its capture by polynucleotide capture probes. The aqueous medium can be merged with a lysis buffer before, during or after emulsification in the oil phase.

Once the emulsion microdroplets are formed, they can be stabilized to prevent coalescence. This can be achieved by adding additional surfactants or polymers that enhance the stability of the droplets. Using these surfactants and microfluidic devices, one can create stable water-in-oil emulsions with precise control over the encapsulation of single cells and beads.

In the single-cell microdroplet, immobilized antibodies 414 on the cell surface can be released by cleaving the cleavable linker, such as by action of a TEV protease 416. The antibody-TdSPH then cleaves and links to a protein capture probe 430 through HUH-tag recognition site. The HUH-tag recognition site can be placed upstream at the 5′ end of the identifier. As a result, barcoded antibodies 432 are released into the lysis buffer once the HUH-tag of TdSPH recognizes, cleaves, and forms a covalent bond with the DNA barcode. This allows capture of transcripts 434 on the bead by polynucleotide capture probes 436 comprising poly(dT) as a polynucleotide capture tag and the barcoded antibody 432 in the supernatant.

### Incubating the Single-Cell Emulsion

The present methods can include a step of incubating a single-cell emulsion microdroplet so that the protein capture tag binds to or forms a conjugate with the expressed protein and the polynucleotide capture tag binds to or forms a conjugate with the nucleic acids from the cell. “Incubating” as used herein refers to any technique or condition that promotes or permits the targeting groups to bind their targets, including but not limited to, maintaining or adjusting temperature or maintaining an emulsion for a period of time which can be predetermined or selected based on other conditions or factors. In some embodiments, after lysing of cells, the aqueous microdroplets within the emulsion are cooled to allow the polynucleotide capture probes to bind to mRNA transcripts from the cell.

The present methods can include a step of detaching the protein capture probes from the solid supports. The detachment can be done after the incubating period and before or after the emulsion is broken.

### Breakage Of Emulsion And Recovery Of Barcoded Expressed Proteins

After a suitable incubating period, the emulsion is broken and the aqueous mixture comprising the solid supports and aqueous medium is collected. After the emulsion is broken, an aqueous mixture remains and it comprises the expressed proteins barcoded with the cell-specific identifiers. For example, the emulsification comprising the microdroplets 424 can be broken using phase inversion. First, the emulsion is centrifuged and the oil layer is discarded to obtain a solution mostly containing the aqueous phase. Then, by adding more aqueous medium (e.g., than three times the volume of the lysis buffer), phase inversion destabilizes the surface tension of the emulsion maintained by excess oils. In some embodiments, emulsion breakage though phage inversion can be enhanced by adding a detergent, such as 0.1-1% Tween-20, to the lysis reagent 428. The emulsions break down, and the solid supports can be separated, such as by using magnetic force to separate magnetic beads. The barcoded antibodies 432 from the aqueous phase can be purified from the medium, such as by passing through a protein G chromatography column.

The collected solid supports can be resuspended in a liquid medium for processing of captured polynucleotides. For example, when the captured polynucleotides are mRNA transcripts, they can be processed by reverse transcription or other amplification techniques to form cDNAs. In some embodiments, the captured mRNA transcripts are processed by overlap extension (OE) RT-PCR in an emulsion to generate cDNAs complementary to mRNA transcripts of interest together. Nested PCR and sequencing of the mRNA transcripts can be performed. In some embodiments, the aqueous medium comprising the cells comprises reverse transcription reagents. In some embodiments, the aqueous medium comprising the cells comprises at least one of polymerase chain reaction and reverse transcriptase polymerase chain reaction reagents. In some embodiments, restriction and ligation may be used to link cDNA of multiple transcripts of interest. In other embodiments, recombination may be used to link cDNA of multiple transcripts of interest.

In some embodiments, the present methods can also comprise a step of detaching the polynucleotide capture probes from the solid supports before reverse transcription or other amplification of the captured polynucleotides.

FIG. 4B shows a distinct band of the expected size corresponding to the cell identifier barcode sequence, with no evidence of non-specific amplification, as confirmed by DNA agarose gel electrophoresis, confirming successful amplification of the cell identifier barcode sequence. The cell-specific identifier 440 attached to the expressed antibody 432 can be amplified using forward and reverse primers 442, 444, and the resulting amplicons can be sequenced.

Additionally, the mRNA captured on the beads is emulsified again. The beads are resuspended in a reverse transcription PCR mixture and applied to oil in the dispersing tube. Homogenization in the dispersing tube induces emulsification of beads into single bead droplets, allowing us to perform reverse transcription overlap extension PCR to amplify the barcoded transcript. We also experimentally confirmed the successful amplification.

The present methods can further comprise separating the solid supports from a surrounding medium after capture of nucleic acids from a cell. By using the capture probes on the solid supports, nucleic acids can be efficiently separated from a mixture. For example, when the solid support is a magnetic bead, it can be separated from a mixture by any suitable separation technique, such as by applying a magnetic field, applying vacuum filtration and/or centrifugation, or any combination thereof. In some embodiments, the solid support is a magnetic bead, and the separation is performed using a magnet or magnetic device.

### Screening of Barcoded Expressed Proteins

The expressed proteins having a cell-specific identifier (i.e., the barcoded expressed proteins) 432 can be used in a wide variety of in vivo or in vitro assays. For example, the barcoded expressed proteins 432 can be used for in vivo assays, by injecting into an animal model to screen for specific or enhanced functionalities, such as extended half-life, tissue localization, solid tumor penetration, enhanced transcytosis through the blood-brain barrier, and transfer through the fetal membrane. For example, to screen for blood-brain barrier permeability, barcoded antibodies that cross the barrier can be pooled, and their barcodes amplified. This allows identification of antibody sequences with enhanced permeability, which could be used for delivery of therapeutic antibodies to the brain to treat neurodegenerative diseases or brain cancer.

As another example, the expressed proteins having a cell-specific identifier can be used for screening the protein with respect to difficult targets such as membrane proteins. Membrane proteins are major drug targets, and antibodies with agonistic or antagonistic functions against these receptors have high therapeutic potential. However, membrane proteins are difficult to express in soluble form and may need the membrane or cell surface for stabilization. Conventional display-based antibody screening is not suitable for membrane proteins due to non-specific binding and the large size of the mediator needed for phenotype-genotype linkage. Using barcoded antibodies, one can more easily screen for antibodies that have agonistic or antagonistic functions targeting membrane proteins.

As yet another example, the expressed proteins having a unique cell-specific identifier can be used for evaluating developability of the protein as a therapeutic, diagnostic, enzyme, or other use. For example, some protein therapeutics tend to form aggregates or denature, leading to inflammatory responses or reduced efficacy. Developability, which refers to the physicochemical and biochemical stability and compatibility of a protein as a drug, is a critical factor in drug development. Using expressed proteins having a unique cell-specific identifier, one can more easily examine and screen for stable, developable protein therapeutics.

After obtaining the amplicons of the cell-specific identifiers from the barcoded expressed protein 432 and of the transcripts with the cell-specific identifiers, they can be analyzed by nucleic acid sequencing. By using the cell-specific identifier as an index, similar to a name tag on a person, the barcoded antibody can be screened and then its sequence can be found by matching it to the sequence of the mRNA associated with the same transcript cell-specific identifier. This enables functional screening of antibodies having identifiers in many ways that were challenging with conventional display-based screening systems.

### Proteins of Interest, Variants, and Libraries

The present method comprises providing a cell that expresses a protein of interest. In some embodiments, the expressed protein is a soluble protein released from the biological cell. The present methods can be used to facilitate analysis of a plurality of cells that express one or more proteins of interest. In some embodiments, the plurality of cells can comprise cells expressing variants of the protein of interest. In such embodiments, the step of providing a solid support can comprise providing a plurality of solid supports, wherein the cell-specific identifier of the protein capture probes and polynucleotide capture probes attached to each of the solid supports is substantially unique in the plurality of solid supports.

The protein of interest can be any protein which is expressed by a cell and which can have one or more variants of interest. “Protein”, as used herein, includes fragments and domains of known and/or unknown (yet-to-be discovered) proteins, including functional domains such as enzymatic domains, binding domains, inhibitory domains, structural domains. “Protein” also includes structural portions, such as folds, turns and loops. In other words, the expressed protein in the present methods can be a domain or portion of a protein of interest. In addition, “protein” as used herein includes oligopeptides, peptides, and protein variants, including naturally-occurring and non-naturally occurring protein analogs and derivatives.

Examples of proteins of interest include, but are not limited to, therapeutic, diagnostic, industrial and experimental or de novo proteins, including ligands, cell surface receptors, antigens, antibodies, cytokines, hormones, transcription factors, signaling modules, cytoskeletal proteins and enzymes. Examples of enzymes for screening by the present methods include, but are not limited to, proteases, carbohydrases, lipases, isomerases, kinases, phosphatases, transferases, and reductases.

The present methods are particularly advantageous with a library of cells producing variants of a protein of interest. In some embodiments, the plurality of cells is a library of cells recombinantly engineered to express variants of the protein of interest. In some embodiments, the library of cells naturally produces variants of the protein of interest. In some embodiments, the library is a library of antibody producing cells such as lymphocytes, B cells, plasma cells, hybridomas, or other cells than naturally or are recombinantly engineered or altered. In some embodiments, the library of antibody producing cells produces antibodies having variant Fc domains. In some embodiments, the library of antibody producing cells produces antibodies having variant Fab domains, such as variant antigen binding domains having one or more changes to complementarity determining regions (CDRs).

Variants of a protein typically have one or more differences, such as substitutions, additions, or deletions of amino acids in the native protein. Variants of a protein of interest can be designed or synthesized by various techniques, including site-directed mutagenesis, combinatorial cloning and random mutagenesis. Additional information about generation a library of variants of a protein can be found in Patten et al. U.S. Pat. No. 6,303,344 B1; Gallo et al. US 20170334970 A1; Haurum et al. US 20060275766 A1; and Dahiyat et al. U.S. Pat. No. 7,315,786 B2.

In some embodiments, the protein of interest has a molecular weight of at least about 10 kDa, or at least about 50 kDa, or at least about 100 kDa, or at least about 150 kDa; alternatively or additionally, the protein of interest has a molecular weight of at most about 500 kDa, or at most about 300 kDa, or at most about 200 kDa; it is contemplated that any of the foregoing minimums and maximums can be combined to form a range, so long as the minimum is smaller than the maximum.

In some embodiments, the protein of interest is or comprises a full-length antibody such as an antibody having two light chains comprising variable and constant regions and two heavy chains comprising one variable and three constant regions.

In some embodiments, the protein of interest is or comprises an antibody Fc domain. In some embodiments, a library of proteins of interest comprises variants of Fc domains having one or more differences relative to a natural Fc domain or other reference Fc domain, which may or may result in variants that bind with greater or lesser affinity to one or more Fc receptors or complement component C1q. Fc receptors include any member of the family of proteins that bind the IgG antibody Fc domain and are substantially encoded by the FcγR genes. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2), as well as any undiscovered human FcγRs or FcγR isoforms or allotypes. Additional information about variants of Fc domains can be found in Lazar et al. US20130156754A1; Stavenhagen et al. US20110305714A1; Maynard et al. WO2023108117A2. Accordingly, the present methods can further comprise screening a library of expressed Fc domains for binding properties (such as greater than or lesser than those of a natural Fc domain or other reference Fc domain) with respect to one or more of Fc receptors.

In some embodiments, the protein of interest is or comprises an antibody variable domain. In some embodiments, the protein of interest is or comprises a bi- or multi-specific antibody.

In some embodiments, the plurality of cells comprises more than 10,000 unique cells, or more than 20,000 unique cells, or more than 50,000 unique cells, or more than 100,000 unique cells, wherein each unique cell is genetically distinct from each other unique cell.

In some embodiments, the library of variants comprises a plurality of different cells which encode different variants of an expressed protein. In some embodiments, the library has at least 2, at least 5, at least 10, at least 50, at least 100, at least 1000, at least 10,000, at least 100,000, at least 1,000,000, at least 10⁷, at least 10⁸, at least 10⁹, at least 10¹⁰ or at least 10¹¹ different cells.

### Expressed Protein Tags And Intermediary Tags

In some embodiments, one or more tags are employed to facilitate capture of an expressed protein and/or a nucleic acid by the capture probes. For example, an expressed protein can have an expressed protein tag attached to it. The expressed protein tag can be attached to the protein after its expression, or it can be a peptide expressed contiguously with the protein. The expressed protein tag can be adapted for binding to the protein capture tag, or to a protein capture tag attached to the protein capture tag. Alternatively the expressed protein tag can be adapted for binding to an intermediary tag, which is itself adapted for binding to the protein capture tag or to a second intermediary tag attached to the protein capture tag. As yet another alternative, a protein capture tag can be adapted for binding to an intermediary tag, or for binding to the expressed protein or a portion thereof. In some embodiments, an expressed protein tag and a protein capture tag are partners or members of a binding pair. In some embodiments, an expressed protein tag and an intermediary tag are partners or members of a first binding pair, and the intermediary tag and the protein capture tag are partners or members of a second binding pair. As used herein, a binding pair comprises two binding partners which specifically and reciprocally bind to each other.

The term “binding pair” as used herein refers to a pair of binding partners that exhibit specific binding between them. In some embodiments, a binding pair can selectively interact through covalent or non-covalent binding. In some embodiments, a binding pair can selectively interact by hybridization, ionic bonding, hydrogen bonding, van der Waals interactions, or any combination of these forces. The term “specific binding” refers to the ability of a binding partner to preferentially bind to its reciprocal binding partner that is present in a homogeneous mixture of different molecules. In some embodiments, specific binding discriminates between a reciprocal binding partner and other molecules by at least 100-fold, 1000-fold, 10,000-fold, 100,000-fold, or more. In some embodiments, the affinity between binding partners of a binding pair when they are specifically bound in a complex is characterized by a K_{D} (dissociation constant) of less than 10⁻⁶ M, less than 10⁻⁷ M, less than 10⁻⁸ M, less than 10⁻⁹ M, less than 10⁻¹⁰ M, less than 10⁻¹¹ M, or less than about 10⁻¹² M, or less.

In some embodiments, a binding partner can comprise, for example, a complementary nucleic acid, biotin, avidin, streptavidin, antibodies or antigen binding fragments thereof, antigens, receptors, receptor domains, receptor fragments, or combinations thereof. Examples of binding pairs include biotin:avidin, biotin:streptavidin, antigen:antibody or antigen binding fragment, complementary nucleic acids, SpyCatcher:SpyTag, HUH-tag:HUH-tag recognition site, as well as others set forth herein. Examples of binding pairs which can be utilized as an expressed protein tag and an intermediary tag or a protein capture tag are set forth in Table 2

TABLE 2

| Expressed Protein Tag | Intermediary Tag or Protein Capture Tag |
|---|---|
| SpyTag: AHIVMVDAYKPTK (SEQ ID NO: 7) | SpyCatcher protein |
| Avi-Tag: GLNDIFEAQKIEWHE (SEQ ID NO: 8) | NHS-Biotin via the enzyme BirA |
| FLAG-tag: DYKDDDDK (SEQ ID NO: 9) | anti-FLAG-tag antibody or antigen-binding |
| | fragment |
| HA-tag: YPYDVPDYA (SEQ ID NO: 10) | anti-HA-tag antibody or antigen-binding |
| | fragment |
| His-tag: homopolymer of histidines (e.g., 5 to | nickel or cobalt chelate |
| 10 H) | |
| Myc-tag: EQKLISEEDL (SEQ ID NO: 11) | anti-Myc-tag antibody or antigen-binding |
| | fragment |
| S-tag: KETAAAKFERQHMDS (SEQ ID NO: 12) | anti-S-tag antibody or antigen-binding |
| | fragment |
| SBP-tag: | streptavidin |
| MDEKTTGWRGGHWEGLAGELEQLRAR | |
| LEHHPQGQREP (SEQ ID NO: 13) | |
| Strep-tag II: WSHPQFEK (SEQ ID NO: 14) | streptavidin |
| V5 tag: GKPIPNPLLGLDST (SEQ ID NO: 15) | anti-V5-tag antibody or antigen-binding |
| | fragment |
| Maltose binding protein-tag | amylose agarose |
| TC tag: tetracysteine | FLASH and ReAsH biarsenical compounds |
| Isopeptag: TDKDMTITFTNKKDAE (SEQ ID NO: 16) | pilin-C protein |
| Calmodulin-tag: | Calmodulin protein |
| KRRWKKNFIAVSAANRFKKISSSGAL | |
| (SEQ ID NO: 17) | |

### Cells and Nucleic Acids

The present methods can be used with cells comprising nucleic acid molecules of various types, such as DNA and/or RNA molecules. DNA molecules include genomic DNA (gDNA), mitochondrial DNA, viral DNA, cDNA, cell-free DNA (cfDNA), circulating tumor DNA (ctDNA), cell-free fetal DNA (cffDNA), or synthetic DNA. The DNA can be double-stranded DNA, single-stranded DNA, fragmented DNA, or damaged DNA. The nucleic molecules can include RNA, DNA, or a mixture of RNA and DNA. The RNA molecules can be mRNA, pre-mRNA, tRNA, rRNA, microRNA, snRNA, piRNA, small non-coding RNA, polysomal RNA, intron RNA, pre-mRNA, viral RNA, or cell-free RNA. In some embodiments, the DNA comprises fragmented genomic DNA and the RNA comprises mRNA or pre-mRNA.

Examples of cells for which single-cell analysis of expressed proteins may be desired include neurons, glial cells, germ cells, gametes, embryonic stem cells, pluripotent stem cells (including induced pluripotent stem cells), adult stem cells, cells of a hematopoietic lineage, differentiated somatic cells, microbial cells, cancer cells (including cancer stem cells), cells infected by a microbe, and cells from diseased tissues.

The nucleic acid can comprise a target sequence. The target sequence can be a gene sequence (such as a sequence of an exon, an intron, or a junction), or a regulatory sequence (such as a promoter, an enhancer, a terminator, a silencer, or an untranslated region (UTR)). The target sequence can be previously known, partially known previously, or previously unknown. The target sequence can comprise a chromosome, chromosome arm, or a gene. The gene can be gene associated with a condition, such as a cancer.

In some embodiments, the present methods comprise attaching adaptors to the nucleic acid molecules or to amplicons thereof. Generally an adaptor is attached to at least one strand of a double-stranded DNA molecule, and usually an adaptor can be a molecule that is at least partially double-stranded. An adaptor may be 40 to 150 bases in length, e.g., 50 to 120 bases. An adaptor can be joined to a 5′ end and/or a 3′ end of a nucleic acid molecule. A Y-adaptor is an adaptor that contains a double-stranded region and a single-stranded region in which the opposing sequences are not complementary. The end of the double-stranded region can be joined to target molecules such as double-stranded fragments of genomic DNA, e.g., by via a transposase-catalyzed reaction. Each strand of a double-stranded DNA molecule that has been joined to a Y adaptor is asymmetrically tagged in that it has the sequence of one strand of the Y-adaptor at one end and the other strand of the Y-adaptor at the other end. Amplification of nucleic acid molecules that have been joined to Y-adaptors at both ends results in an asymmetrically tagged nucleic acid, i.e., a nucleic acid that has a 5′ end containing one tag sequence and a 3′ end that has another tag sequence.

Nucleic acid molecules can be denatured to form single-stranded molecules which are then amplified using amplification primers to form double-stranded products, and/or processed by other techniques. In some embodiments, the processing comprises amplifying a nucleic acid, before and/or after it is attached to an adaptor. In some embodiments, an adaptor is located at a 5′-end of a target sequence in a cDNA or an nucleic acid, and the adaptor provides a priming site for amplification of the target sequence.

Nucleic acids can be amplified using any suitable method. In some embodiments, the nucleic acid is amplified using polymerase chain reaction (PCR). In general, PCR comprises denaturation of polynucleotide strands (e.g., DNA melting), annealing of primers to the denatured polynucleotide strand, and extension of primers with a polymerase to synthesize the complementary polynucleotide. The process generally requires a DNA polymerase, forward and reverse primers, deoxynucleoside triphosphates, bivalent cations, and a buffer solution. In some embodiments, the nucleic acid is amplified by linear amplification. In some embodiments, the nucleic acid is amplified using Emulsion PCR, Bridge-PCR, or Rolling Circle amplification. The amplicons of the nucleic acid may be analyzed to determine the order of base pairs using a suitable sequencing method.

### Sequencing Nucleic Acid Amplicons and Identifiers

The present methods provide nucleic acids (such as amplicons of a cell's mRNA, as well as cell-specific identifiers), and those nucleic acids can be sequenced by any suitable technique, such as various next-generation sequencing (NGS) techniques and systems. Sequencing can be performed by massively-parallel sequencing, high-throughput sequencing, pyrosequencing, Sanger sequencing, sequencing-by-ligation, sequencing by synthesis, sequencing-by-hybridization, single molecule sequencing by synthesis (SMSS), Ion Torrent sequencing, shotgun sequencing, single molecule nanopore sequencing, sequencing by ligation, sequencing by hybridization, sequencing by nanopore current restriction, Maxim-Gilbert sequencing, primer walking, or a combination thereof. Additional details regarding various sequencing techniques can be found in Bartha et al. US 20140200147 A1; Christians et al. US 20170275691 A1. Sequencing can be performed by various systems currently available, such as sequencing systems available from by Illumina, Pacific Biosciences, Oxford Nanopore, or Life Technologies.

Next-generation sequencing may provide a plurality of raw genetic data corresponding to the genes or transcripts of a cell, including sequencing reads. A read may include a string of nucleic acid bases corresponding to a sequence of a nucleic acid molecule that has been sequenced. In some situations, systems and methods provided herein may be used with proteomic information.

Many next-generation sequencing (NGS) techniques involve parallel sequencing of a library of nucleic acids by a sequencing instrument. Preparation of a sequencing library generally includes various steps such as amplification of the nucleic acids, attachment of adaptors, and/or other preparatory steps. Emerging polynucleotide sequencing platforms can enable direct detection and analysis of nucleic acid molecules without the need for amplification, though the nucleic acids often require an adaptor or other moiety to immobilize the nucleic acid for sequencing steps. An adaptor can be attached to one or both ends of nucleic acid molecules in order to add sites for primer binding and for immobilization of the nucleic acid on a surface such as a flowcell or a bead, and to add other functional sequences to the fragments. Various kinds of adaptors are used in sequencing preparation kits to add these sites or sequences to the nucleic acids from the sample. Adaptors can be attached in various ways, such as by ligation, primer extension, tagmentation, and other techniques.

A sequencing library can be generated in a variety of ways, with different objectives regarding the nucleic acids to be used as inputs. For instance, PCR can be used with target-specific primers to generate a library of amplicons covering regions of interest in the nucleic acid sample. Other methods of library preparation involve random fragmentation of the nucleic acid sample by enzymatic or physical shearing methods, followed by amplification using common adaptor sequences. Enrichment procedures are used to remove or separate sequences of interest from the rest of the sample.

The present methods may be used in conjunction with a high-throughput sequencing technique to determine sequences of nucleic acids encoding the protein of interest. In some embodiments, a high-throughput sequencing method comprises three steps: library preparation, immobilization, and sequencing. Adaptors are attached to one or both ends of the nucleic acids to form a sequencing library. The sequencing library molecules are immobilized on a solid support, and sequencing reactions are performed to identify the nucleic acid sequence. The high-throughput sequencing method may employ an amplification process to provide colonies or copies of the nucleic acid to be sequenced. In some embodiments, cell-specific barcoded cDNA molecules synthesized from the cell's RNA molecules are sequenced without amplification. In some embodiments, a cDNA molecule is sequenced using a single-molecule sequencing platform.

In some embodiments, cell-specific barcoded cDNA molecules synthesized from captured mRNA molecules (or amplicons thereof) can be further analyzed using various methods including southern blotting, polymerase chain reaction (PCR) (e.g., real-time PCR (RT-PCR), digital PCR (dPCR), droplet digital PCR (ddPCR), quantitative PCR (Q-PCR), nCounter analysis (Nanostring technology), gel electrophoresis, DNA microarray, mass spectrometry (e.g., tandem mass spectrometry, matrix-assisted laser desorption ionization time of flight mass spectrometry (MALDI-TOF MS), chain termination sequencing (Sanger sequencing), or next generation sequencing. The input DNA molecules (or amplicons thereof) can also by analyzed by such methods.

Nucleic acid libraries generated using methods described herein can be generated from more than one sample. Each library can have a different index associated with the sample. For example, a capture probe or an anchor probe can comprise an index that can be used to identify nucleic acids as coming from the same sample (e.g., a first set of capture probes or anchor probes comprising the same first index can be used to generate a first library from a first sample from a first subject, and a second set of capture probes or anchor probes comprising the same second index can be used to generate a second library from a second sample from a second subject, the first and second library can be pooled, sequenced, and an index can be used to discern from which sample a sequenced nucleic acid was derived). Amplified products generated using the methods described herein can be used to generate libraries from at least 2, 5, 10, 25, 50, 100, 1000, or 10,000 samples, each library with a different index, and the libraries can be pooled and sequenced, e.g., using a next generation sequencing technology.

The sequencing can generate at least 100, 1000, 5000, 10,000, 100,000, 1,000,000, or 10,000,000 sequence reads. The sequencing can generate between about 100 sequence reads to about 1000 sequence reads, between about 1000 sequence reads to about 10,000 sequence reads, between about 10,000 sequence reads to about 100,000 sequence reads, between about 100,000 sequence reads and about 1,000,000 sequence reads, or between about 1,000,000 sequence reads and about 10,000,000 sequence reads.

The depth of sequencing can be about 1×, 5×, 10×, 50×, 100×, 1000×, or 10,000×. The depth of sequencing can be between about 1× and about 10×, between about 10× and about 100×, between about 100× and about 1000×, or between about 1000× and about 10000×.

Compositions and Kits for Attaching Cell-Specific Identifiers To Proteins and Nucleic Acids

As another aspect of the present disclosure, compositions and kits are provided which comprise sets of solid supports having protein capture probes and polynucleotide capture probes attached to a surface of the solid support. The compositions and kits are configured for attaching a cell-specific identifier to nucleic acids and expressed proteins of a cell. The compositions can comprise other components such as aqueous medium, buffers, reagents, etc. The kits can comprise the sets of solid supports in one or more vessels, such as vials or tubes. The solid supports and capture probes of the compositions and kits can have any of the features described in more detail with respect to the present methods.

In some embodiments, the present compositions and kits comprise one or more solid supports with oligonucleotides comprising functional sequences configured to be attached to nucleic acids and proteins of interest.

In addition to above-mentioned components, the kits may further include instructions for using the components of the kit to practice the present methods, i.e., to attach cell-specific identifiers to a protein of interest expressed by a cell and nucleic acids from the same cell. The instructions for practicing the present methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, portable drive, or cloud-based storage, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### ADDITIONAL TERMINOLOGY

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

All patents and publications, including all sequences disclosed within such patents and publications, referred to herein are expressly incorporated by reference. The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The present technology may employ, unless otherwise indicated, techniques and descriptions of organic chemistry, polymer technology, molecular biology (including recombinant techniques), cell biology, biochemistry, and immunology, which are within the skill of the art. Such techniques include polymer array synthesis, hybridization, ligation, and detection of hybridization using a label.

As used herein, the singular forms “a”, “an”, and “the” include plural referents unless the context clearly dictates otherwise. For example, the term “a primer” refers to one or more primers, i.e., a single primer and multiple primers. A “plurality” contains at least 2 members. In certain cases, a plurality may have at least 10, at least 100, at least 100, at least 10,000, at least 100,000, at least 10⁶, at least 10⁷, at least 10⁸ or at least 10⁹ or more members.

As used in the specification and the appended claims and in addition to its ordinary meaning, the terms “approximately” and “about” mean to within an acceptable limit or amount to one having ordinary skill in the art. The term “about” generally refers to plus or minus 15% of the indicated number. For example, “about 10” may indicate a range of 8.5 to 11.5. For example, “approximately the same” means that one of ordinary skill in the art considers the items being compared to be the same. It should be understood that any of the values disclosed herein are also a disclosure of the approximate value (e.g., the disclosure of “0.10” shall also constitute a disclosure of “about 0.10”), and any disclosure of an approximate value is a disclosure of the value itself (e.g., the disclosure of “about 0.10” shall also constitute a disclosure of “0.10”), unless the context indicates otherwise.

It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used in the specification and appended claims, and in addition to their ordinary meanings, the terms “substantial” or “substantially” mean to within acceptable limits or degree to one having ordinary skill in the art. For example, “substantially inactive” means that one skilled in the art considers the level of activity to be negligible.

The term “target” as used herein refers to a nucleic acid of interest, or which is desired for sequencing and/or other analysis. One or more targets may be present within an nucleic acid, or a construct or complex made from an nucleic acid. A target may be single-stranded or double-stranded, and often is double-stranded DNA when attached to an adaptor to form a nucleic acid construct. Target as used herein can refer to a specific sequence or the complement thereof or to both. The term target encompasses any nucleic acid molecule of biological or synthetic origin whose sequence or other characteristic is of interest. The target sequence generally does not include identifiers, primer binding regions, or adaptors sequences which may be added to the nucleic acid molecule to prepare an nucleic acid construct for sequencing or other analysis.

The terms “amplifying” and “amplification” as used herein refer to synthesizing nucleic acid molecules that are complementary to one or both strands of an nucleic acid. Amplifying and amplification of a nucleic acid may linear, exponential, or a combination thereof. Non-limiting examples of nucleic acid amplification methods include reverse transcription, primer extension, polymerase chain reaction, ligase chain reaction, helicase-dependent amplification, asymmetric amplification, rolling circle amplification, and multiple displacement amplification. Amplifying a nucleic acid molecule may include the steps of denaturing a double-stranded nucleic acid, annealing primers the nucleic acid at a temperature that is below the melting temperatures of the primers, and enzymatically elongating from the primers to generate an amplification product. The terms “amplicon” or “amplification product” refer to the nucleic acid sequences which are produced from an amplifying process, including the nucleic acid molecules synthesized by amplifying the nucleic acid or its complementary sequence, as well as the nucleic acid molecules synthesized from other amplicons. The denaturing, annealing and elongating steps each can be performed one or more times. Amplification generally does not change the nucleic acid sequence unless errors arise during the amplification. Additional details regarding various amplification techniques can be found in Fan et al. US20230295723A1.

Amplification typically requires the presence of deoxyribonucleoside triphosphates, a DNA polymerase enzyme and an appropriate buffer and/or co-factors for optimal activity of the polymerase enzyme. Reverse transcription is a linear amplification reaction that employs a specialized DNA polymerase (reverse transcriptase) to copy RNA into cDNA (complementary DNA) using deoxyribonucleoside triphosphates.

The term “adaptor” generally refers to a nucleic acid molecule that is attached to an nucleic acid molecule to add a desired structure or function. The term “tag” also generally refers to a moiety that can add a desired structure or function, though it is contemplated that a tag may be a nucleic acid molecule, a molecule other than a nucleic acid, or a combination thereof. For example, a “tag” as used herein can comprise an adaptor conjugated to a non-nucleic acid binding partner such as DIG. As another example, a “tag” as used herein can comprise an antibody conjugated to a biotin moiety. As another example, an adaptor can be attached to an input fragment or an amplicon thereof to add a binding site for a NGS platform. In some embodiments, an adaptor refers to molecules that are at least partially double-stranded. An adaptor or a tag may be any desired length, including but not limited to 40 to 150 bases in length, e.g., 50 to 120 bases, although adaptors and tags outside of this range are envisioned.

The terms “identifier” and “barcode”, as used herein, refer to a sequence of nucleotides which can be used to identify a molecule to which it is attached, e.g., to identify the origin or source of the molecule, such as being from a particular subject, tissue, sample, or cell. The terms “identifier” and “barcode” are used interchangeably herein, unless the context indicates otherwise. Identifiers have been commonly used as sample indices or sample barcodes, where the same sequence is unique for all nucleic acids from a particular sample. Sample barcodes enable the mixing of nucleic acids from different samples in one sequencing run, as the different sample barcode sequences enable the correct assignment of sequencing reads to each sample. One, two, or more sample barcodes may be used.

Identifiers also comprise molecular barcodes (MBCs) or unique molecular identifier (UMI) sequences, which function to identify amplicons or copies from an individual nucleic acid molecule within a sample. UMIs may comprise random nucleotides, known nucleotides, or a mixture of random and known nucleotides. UMIs enable more accurate sequencing by allowing error correction of sequences and more accurate estimation of the original number of nucleic acids. Molecular barcodes called degenerate base regions (DBR) are disclosed in U.S. Pat. No. 8,481,292 (Population Genetics Technologies Ltd.). The DBRs are random sequence tags that are attached to molecules that are present in the sample. DBRs and other molecular barcodes allow one to distinguish PCR errors during sample preparation from mutations and other variants that were present in the original nucleic acid.

“Cell-specific identifiers” are different from both sample barcodes and UMIs in that they are adapted for identifying multiple molecules from a specific cell, as opposed to molecules from multiple cells within a sample, or an individual molecule from a cell or a sample.

Identifiers or barcodes may be added to one strand of an nucleic acid (e.g., to a single-stranded DNA molecule or an RNA molecule), or to both strands of one end of a double-stranded nucleic acid (both the 5′ end of the + strand, and the 3′ end of the − strand in the duplex), or to both ends of a double-stranded nucleic acid (e.g., to both the 5′ and the 3′ ends of both the + and the − strands of the duplex).

The term “nucleotide” as used herein refers to a phosphate ester of a nucleoside, wherein the esterification site typically corresponds to the hydroxyl group attached to the C-5 position of the pentose sugar. In some cases nucleotides comprise nucleoside polyphosphates. However, the terms “added nucleotide,” “incorporated nucleotide,” “nucleotide added” and “nucleotide after incorporation” all refer to a nucleotide residue that is part of an oligonucleotide or polynucleotide chain.

The term “nucleotide” refers to naturally-occurring nucleotides including guanine, cytosine, adenine, thymine, uracil (G, C, A, T and U respectively), as well as modified pyrimidine and purine derivatives and other non-naturally occurring moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleotides also include modifications on the sugar moiety, e.g., wherein one or more of the hydroxyl groups are replaced with halogen atoms or aliphatic groups, are functionalized as ethers, amines, or the likes.

The term “oligonucleotide”, “nucleic acid” and “polynucleotide” are used interchangeably herein to describe a nucleotide-containing polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced naturally, chemically, enzymatically or synthetically. In some contexts, such for capture probes, the terms include polymers having PNA, LNA or UNA. DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylene carbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid”, or “UNA”, is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively.

The terms “nucleoside”, “nucleotide”, “deoxynucleoside”, and “deoxynucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the “nucleoside”, “nucleotide”, “deoxynucleoside”, and “deoxynucleotide” include those moieties that contain not only conventional ribose and deoxyribose sugars, but other sugars as well. Modified nucleosides, nucleotides, deoxynucleosides or deoxynucleotides also include modifications on the sugar moiety, e.g., wherein one or more of the hydroxyl groups are replaced with halogen atoms or aliphatic groups, or are functionalized as ethers, amines, or the like.

Natural nucleotides or nucleosides are defined herein as adenine (A), thymine (T), guanine (G), and cytosine (C). It is recognized that certain modifications of these nucleotides or nucleosides occur in nature. However, modifications of A, T, G, and C that occur in nature that affect hydrogen bonded base pairing are considered to be non-naturally occurring. For example, 2-aminoadenosine is found in nature, but is not a “naturally occurring” nucleotide or nucleoside as that term is used herein. Other non-limiting examples of modified nucleotides or nucleosides that occur in nature that do not affect base pairing and are considered to be naturally occurring are 5-methylcytosine, 3-methyladenine, 0(6)-methylguanine, and 8-oxoguanine, etc. Nucleotides include any nucleotide or nucleotide analog, whether naturally-occurring or synthetic. Exemplary nucleotides include phosphate esters of deoxyadenosine, deoxycytidine, deoxyguanosine, deoxythymidine, adenosine, cytidine, guanosine, and uridine. Other nucleotides include an adenine, cytosine, guanine, thymine base, a xanthine or hypoxanthine, 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methylRNA, peptide nucleic acids, modified peptide nucleic acids, locked nucleic acids and any other structural moiety that can act substantially like a nucleotide or base, for example, by exhibiting base-complementarity with one or more bases that occur in DNA or RNA and/or by being capable of base-complementary incorporation, and includes chain-terminating analogs. A nucleotide corresponds to a specific nucleotide species if they share base-complementarity with respect to at least one base.

In addition to purines and pyrimidines, modified nucleotides or analogs, as those terms are used herein, include any compound that can form a hydrogen bond with one or more naturally occurring nucleotides or with another nucleotide analog. Any compound that forms at least two hydrogen bonds with T or with a derivative of T is considered to be an analog of A or a modified A. Similarly, any compound that forms at least two hydrogen bonds with A or with a derivative of A is considered to be an analog of T or a modified T. Similarly, any compound that forms at least two hydrogen bonds with G or with a derivative of G is considered to be an analog of C or a modified C. Similarly, any compound that forms at least two hydrogen bonds with C or with a derivative of C is considered to be an analog of G or a modified G. It is recognized that under this scheme, some compounds will be considered for example to be both A analogs and G analogs (purine analogs) or both T analogs and C analogs (pyrimidine analogs).

The term “antibody” is used interchangeably herein with “immunoglobulin” Those terms refer to a protein consisting of one or more polypeptides that specifically binds an antigen. One example of an antibody is the naturally occurring structural unit found in humans and other mammals which comprises a tetramer of two identical pairs of antibody chains, each pair having one light and one heavy chain. In each pair, the light and heavy chain variable regions are together responsible for binding to an antigen, and the constant regions are responsible for the antibody effector functions. The term antibody encompasses monoclonal antibodies, polyclonal antibodies, chimeric antibodies, humanized antibodies, human antibodies, murine antibodies, rabbit antibodies, camelid antibodies, and antibodies from other mammalian and non-mammalian species. The term antibody also encompasses single-chain antibodies, bi-specific hybrid antibodies, and fusion proteins comprising an antigen-binding portion of an antibody and a non-antibody protein. The term antibody also encompasses includes antigen-binding fragments of antibodies which retain specific binding to antigen, including, but not limited to, Fab, Fv, scFv, and Fd fragments.

The term “partition,” as used herein, refers to a space or volume that may be suitable to contain one or more species or conduct one or more reactions. A partition may be a physical compartment, such as a droplet or well. The partition may isolate space or volume from another space or volume. A droplet may be a first phase (e.g., aqueous medium) in a second phase (e.g., oil) immiscible with the first phase. Alternatively, a droplet may be a first phase in a second phase that does not phase separate from the first phase, such as, for example, a capsule or liposome in an aqueous medium. A partition may comprise one or more other (inner) partitions. In some cases, a partition may be a virtual compartment that can be defined and identified by an index across multiple and/or remote physical compartments. For example, a physical compartment may comprise a plurality of virtual compartments.

### Exemplary Embodiments

Embodiment A1. A method for attaching a cell-specific identifier to a protein of interest expressed by a cell and to a nucleic acid from the cell encoding the protein. The method comprises providing a cell that expresses a protein; providing a solid support having protein capture probes and polynucleotide capture probes attached to a surface of the solid support. The protein capture probes and the polynucleotide capture probes on the solid support comprise a cell-specific identifier; the protein capture probe comprises a protein capture tag, and the polynucleotide capture probe comprises a polynucleotide capture tag. The protein capture tag binds to or forms a conjugate with the expressed protein, and the polynucleotide capture tag binds to or forms a conjugate with a nucleic acid encoding the expressed protein. The method also comprises forming a single-cell emulsion comprising the solid support and the cell; incubating the single-cell emulsion so that the protein capture tag binds to or forms a conjugate with the expressed protein and the polynucleotide capture tag binds to or forms a conjugate with the nucleic acid; and detaching the protein capture probe from the solid support.

Embodiment A2. The method of embodiment A1, wherein the step of providing a cell comprises providing a plurality of cells expressing the protein of interest; and the step of providing a solid support comprises providing a set of solid supports, wherein the cell-specific identifier of each of the solid supports is substantially unique in the set of solid supports.

Embodiment A3. The method of embodiment A2, wherein the plurality of cells comprises cells expressing variants of the protein of interest.

Embodiment A4. The method of embodiment A2, wherein the plurality of cells is a library of cells recombinantly engineered to express variants of the protein of interest.

Embodiment A5. The method of any of embodiments A2 to A4, wherein the plurality of cells comprises antibody-producing cells.

Embodiment A6. The method of embodiment A5, wherein the antibody-producing cells produce antibodies having variant Fc domains.

Embodiment A7. The method of embodiment A5, wherein the antibody-producing cells produce antibodies having variant Fab domains.

Embodiment A8. The method of any of embodiments A2 to A7, wherein the plurality of cells comprises more than 10,000 unique cells, wherein each unique cell expresses a different variant of the protein of interest.

Embodiment A9. The method of any of embodiments A1 to A8, wherein the expressed protein is secreted by the cell.

Embodiment A10. The method of any of embodiments A1 to A9, further comprising providing an expressed protein tag attached to the protein of interest.

Embodiment A11. The method of embodiment A10, wherein the protein expressed by the cell is a fusion of the protein of interest and an expressed protein tag.

Embodiment A12. The method of embodiment A10, wherein the expressed protein tag is avi-tag or SpyTag.

Embodiment A13. The method of embodiment A10, further comprising attaching an intermediary tag to the expressed protein tag to produce a dual tagged construct, wherein the intermediary tag comprises a reciprocal binding partner of the expressed protein tag.

Embodiment A14. The method of embodiment A13, wherein the intermediary tag further comprises a binding partner of a second binding pair and the protein capture tag comprises a reciprocal binding partner of the second binding pair, with the proviso that the binding partner of the second binding pair is not the binding partner or the reciprocal binding partner of the first binding pair.

Embodiment A15. The method of embodiment A14, wherein the binding partner of the second binding pair is selected from HUH-tag and an avidin moiety.

Embodiment A16. The method of embodiment A15, wherein a reciprocal binding partner of the second binding pair is selected from a HUH-tag recognition site and biotin.

Embodiment A17. The method of any of embodiments A1 to A14, wherein the protein capture tag comprises a HUH-tag recognition site, and the method further comprises binding an intermediary tag comprising a HUH-tag to the expressed protein of interest.

Embodiment A18. The method of embodiment A17, wherein the expressed protein of interest comprises SpyTag as an expressed protein tag, and the intermediary tag is a tandem SpyCatcher-HUH-tag construct.

Embodiment A19. The method of any of embodiments A1 to A11, wherein the protein capture tag comprises an amine-binding moiety that binds a lysine residue of the protein of interest or of an expressed protein tag.

Embodiment A20. The method of any of embodiments A1 to A19, further comprising introducing a lysis reagent into the single-cell emulsion, thereby inducing lysis of the cell and releasing nucleic acid from the cell.

Embodiment A21. The method of embodiment A1, wherein the protein capture tag covalently binds to the expressed protein, to an expressed protein tag, or to an intermediary tag.

Embodiment A22. The method of any of embodiments A1 to A21, wherein the released nucleic acid comprises mRNA, and the polynucleotide capture tag comprises poly(dT).

Embodiment A23. The method of any of embodiments A1 to A22, wherein the identifier is a non-contiguous assembly of barcodes.

Embodiment A24. The method of embodiment A23, further comprising synthesizing the cell-specific identifiers by a split-and-pool synthesis method, which ligates DNA barcode segments step-by-step.

Embodiment A25. The method of any of embodiments A1 to A22, wherein the protein capture probes and the polynucleotide capture probes are orthogonally synthesized.

Embodiment A26. The method of embodiment A1, wherein protein capture tag non-covalently binds to the expressed protein, to an expressed protein tag, or to an intermediary tag.

Embodiment B1. A composition for attaching a cell-specific identifier to a protein expressed by a cell and to a nucleic acid from the cell encoding the expressed protein. The composition comprises a solid support having a protein capture probe and a polynucleotide capture probe attached to a surface of the solid support, wherein: the protein capture probe and the polynucleotide capture probe on the solid support comprise a cell-specific identifier; and the protein capture probe comprises a protein capture tag, and the polynucleotide capture probe comprises a polynucleotide capture tag.

Embodiment B2. The composition of embodiment B1, wherein the composition comprises a set of the solid supports, and the cell-specific identifier of each of the solid supports of the set is substantially unique.

Embodiment B3. The composition of embodiment B1 or B2, wherein the protein capture probe is attached to the solid support by a cleavable linker.

Embodiment B4. The composition of embodiment B1 or B2, wherein the protein capture probe is attached to the solid support by a sequence comprising a HUH-tag recognition site.

Embodiment B5. The composition of any of embodiments B2 to B4, wherein the set of solid supports comprises more than 10,000 unique solid supports, wherein each unique solid support has a unique cell-specific identifier.

Embodiment B6. The composition of any of embodiments B1 to B5, wherein the protein capture tag comprises a HUH-tag recognition site, and the method further comprises binding an intermediary tag comprising a HUH-tag to the expressed protein of interest.

Embodiment B7. The composition of any of embodiments B1 to B6, wherein the intermediary tag is a tandem SpyCatcher-HUH-tag construct.

Embodiment B8. The composition of embodiment B7, wherein the expressed protein tag is a SpyTag.

Embodiment B9. The composition of any of embodiments B1 to B8, wherein the protein capture tag comprises an amine-binding moiety that binds a lysine residue of the protein of interest or of an expressed protein tag.

Embodiment B10. The composition of embodiment B1, wherein the protein capture tag covalently binds to the expressed protein, to an expressed protein tag, or to an intermediary tag.

Embodiment B11. The composition of embodiment B1, wherein protein capture tag non-covalently binds to the expressed protein, to an expressed protein tag, or to an intermediary tag.

Embodiment B12. The composition of any of embodiments B1 to B1, wherein the polynucleotide capture tag comprises poly(dT).

Embodiment B13. The composition of any of embodiments B1 to B12, wherein the identifier is a non-contiguous assembly of barcodes.

Embodiment B14. The composition of any of embodiments B1 to B13, wherein the solid support is a magnetic bead.

Embodiment B15. The composition of any of embodiments B1 to B14, further comprising an aqueous medium in which the solid support is suspended.

Embodiment B16. The composition of embodiment B15, wherein the composition comprises a lysis reagent.

Embodiment B17. A method for attaching a cell-specific identifier to a protein of interest expressed by a cell and to a nucleic acid from the cell encoding the protein, using the composition of any of Embodiments B1 to B16.

Embodiment C1. A method of synthesizing a solid support for attaching a cell-specific identifier to a protein expressed by a cell and to a nucleic acid from the cell. The method comprises providing a solid support having a linking moiety on a surface; attaching a protein capture probe and a polynucleotide capture probe to a surface of the solid support. The protein capture probe and the polynucleotide capture probe on the solid support comprise a cell-specific identifier; and the protein capture probe comprises a protein capture tag, and the polynucleotide capture probe comprises a polynucleotide capture tag.

Embodiment C2. The method of embodiment C1, wherein the solid support is provided as a set of the solid supports, and the method comprises splitting the set of solid supports into first partitions; adding an oligonucleotide to the first partitions, wherein different oligonucleotides are added to different partitions; ligating the added oligonucleotides to an end of the capture probes in the first partitions; pooling the set of solid supports; partitioning the set of solid supports into second partitions.

Embodiment C3. The method of embodiment C1 or C2, further comprising repeating the adding and ligating steps one or more times, thereby synthesizing unique cell-specific identifiers on each of the solid supports in the set.

Embodiment C4. The method of any of embodiments C1 to C3, wherein the oligonucleotides comprise a 5′ end, an identifier segment, and a 3′ end.

Embodiment C5. The method of embodiment C4, wherein the 5′ end and the 3′ end are configured for orthogonal synthesis of protein capture probes and polynucleotide capture probes.

In view of this disclosure it is noted that the methods and kits can be implemented in keeping with the present teachings. Further, the various components, materials, structures and parameters are included by way of illustration and example only and not in any limiting sense. In view of this disclosure, the present teachings can be implemented in other applications and components, materials, structures and equipment to implement these applications can be determined, while remaining within the scope of the appended claims.

We claim:

1. A method for attaching a cell-specific identifier to a protein of interest expressed by a cell and to a nucleic acid from the cell encoding the protein, the method comprising:
providing a cell that expresses a protein;
providing a solid support having protein capture probes and polynucleotide capture probes attached to a surface of the solid support, wherein:
the protein capture probes and the polynucleotide capture probes on the solid support comprise a cell-specific identifier;
the protein capture probe comprises a protein capture tag, and the polynucleotide capture probe comprises a polynucleotide capture tag, wherein the protein capture tag binds to or forms a conjugate with the expressed protein, and the polynucleotide capture tag binds to or forms a conjugate with a nucleic acid encoding the expressed protein;
forming a single-cell emulsion comprising the solid support and the cell;
incubating the single-cell emulsion so that the protein capture tag binds to or forms a conjugate with the expressed protein and the polynucleotide capture tag binds to or forms a conjugate with the nucleic acid; and
detaching the protein capture probe from the solid support.

2. The method of claim 1, wherein the step of providing a cell comprises providing a plurality of cells expressing the protein of interest; and
the step of providing a solid support comprises providing a set of solid supports, wherein the cell-specific identifier of each of the solid supports is substantially unique in the set of solid supports.

3. The method of claim 2, wherein the plurality of cells comprises cells expressing variants of the protein of interest.

4. The method of claim 2, wherein the plurality of cells is a library of cells recombinantly engineered to express variants of the protein of interest.

5. The method of claim 2, wherein the plurality of cells comprises antibody-producing cells.

6. The method of claim 5, wherein the antibody-producing cells produce antibodies having variant Fc domains.

7. The method of claim 5, wherein the antibody-producing cells produce antibodies having variant Fab domains.

8. The method of claim 2, wherein the plurality of cells comprises more than 10,000 unique cells, wherein each unique cell expresses a different variant of the protein of interest.

9. The method of claim 1, wherein the expressed protein is secreted by the cell.

10. The method of claim 1, further comprising providing an expressed protein tag attached to the protein of interest.

11. The method of claim 10, wherein the protein expressed by the cell is a fusion of the protein of interest and an expressed protein tag.

12. The method of claim 10, wherein the expressed protein tag is avi-tag or SpyTag.

13. The method of claim 10, further comprising attaching an intermediary tag to the expressed protein tag to produce a dual tagged construct, wherein the intermediary tag comprises a reciprocal binding partner of the expressed protein tag.

14. The method of claim 13, wherein the intermediary tag further comprises a binding partner of a second binding pair and the protein capture tag comprises a reciprocal binding partner of the second binding pair,
with the proviso that the binding partner of the second binding pair is not the binding partner or the reciprocal binding partner of the first binding pair.

15. The method of claim 14, wherein the binding partner of the second binding pair is selected from HUH-tag and an avidin moiety.

16. The method of claim 15, wherein a reciprocal binding partner of the second binding pair is selected from a HUH-tag recognition site and biotin.

17. The method of claim 1, wherein the protein capture tag comprises a HUH-tag recognition site, and the method further comprises binding an intermediary tag comprising a HUH-tag to the expressed protein of interest.

18. The method of claim 17, wherein the expressed protein of interest comprises SpyTag as an expressed protein tag, and the intermediary tag is a tandem SpyCatcher-HUH-tag construct.

19. A composition for attaching a cell-specific identifier to a protein expressed by a cell and to a nucleic acid from the cell encoding the expressed protein, the composition comprising:
a solid support having a protein capture probe and a polynucleotide capture probe attached to a surface of the solid support, wherein:
the protein capture probe and the polynucleotide capture probe on the solid support comprise a cell-specific identifier; and
the protein capture probe comprises a protein capture tag, and the polynucleotide capture probe comprises a polynucleotide capture tag.

20. A method of synthesizing a solid support for attaching a cell-specific identifier to a protein expressed by a cell and to a nucleic acid from the cell, comprising:
providing a solid support having a linking moiety on a surface;
attaching a protein capture probe and a polynucleotide capture probe to a surface of the solid support, wherein:
the protein capture probe and the polynucleotide capture probe on the solid support comprise a cell-specific identifier; and
the protein capture probe comprises a protein capture tag, and the polynucleotide capture probe comprises a polynucleotide capture tag.


SEQUENCE LISTING (17 sequences)

SEQ ID NO: 1 - DNA, 15 bp, synthetic construct
  source 1..15: mol_type = other DNA
aagtattaccagaaa

SEQ ID NO: 2 - DNA, 23 bp, synthetic construct
  source 1..23: mol_type = other DNA
tgcttccgtactacgacccccca

SEQ ID NO: 3 - DNA, 22 bp, synthetic construct
  source 1..22: mol_type = other DNA
tttgcgtggggtgtggtgcttt

SEQ ID NO: 4 - DNA, 38 bp, synthetic construct
  source 1..38: mol_type = other DNA
ccagtttctcgaagagaaaccggtaagtgcaccctccc

SEQ ID NO: 5 - DNA, 45 bp, synthetic construct
  source 1..45: mol_type = other DNA
acgcgaacggaacgttcgcataagtgcgcccttacgggatttaac

SEQ ID NO: 6 - protein, 15 aa, synthetic construct
GLNDIFEAQKIEWHE

SEQ ID NO: 7 - protein, 13 aa, synthetic construct
AHIVMVDAYKPTK

SEQ ID NO: 8 - protein, 15 aa, synthetic construct
GLNDIFEAQKIEWHE

SEQ ID NO: 9 - protein, 8 aa, synthetic construct
DYKDDDDK

SEQ ID NO: 10 - protein, 9 aa, synthetic construct
YPYDVPDYA

SEQ ID NO: 11 - protein, 10 aa, synthetic construct
EQKLISEEDL

SEQ ID NO: 12 - protein, 15 aa, synthetic construct
KETAAAKFERQHMDS

SEQ ID NO: 13 - protein, 37 aa, synthetic construct
MDEKTTGWRGGHWEGLAGELEQLRARLEHHPQGQREP

SEQ ID NO: 14 - protein, 8 aa, synthetic construct
WSHPQFEK

SEQ ID NO: 15 - protein, 14 aa, synthetic construct
GKPIPNPLLGLDST

SEQ ID NO: 16 - protein, 16 aa, synthetic construct
TDKDMTITFTNKKDAE

SEQ ID NO: 17 - protein, 26 aa, synthetic construct
KRRWKKNFIAVSAANRFKKISSSGAL